(19) United States
(12) Patent Application Publication
(10) Pub. No.: US 2009/0318873 A1
(43) Pub. Date: Dec. 24, 2009

(54) MEDICAL MALECOT WITH MAGNETS

(75) Inventor: F. Kristen Bailey, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/145,161

(22) Filed: Jun. 24, 2008

(51) Int. Cl. A61M 5/14

(52) U.S. Cl. 604/177

(57) ABSTRACT

Embodiments of a malecot device may include magnets configured to maintain a deployed configuration of malecot wings of the device. A malecot device may also include a deployment member that is removably attached thereto.

### TECHNICAL FIELD

The present patent application relates generally to medical devices, and particularly to a malecot device suitable for medical or non-medical applications.

### BACKGROUND

Malecots are well-known within the medical art. In particular, they are known for use in catheter retention applications and other applications where it is desirable to have an expandable member to hold a tubular device within a patient body. For example, malecots are known for use in securing drainage tubes and feeding tubes. As shown in FIG. 1A, an existing malecot design 100 typically includes an outer sheath 102 having a distal portion that is affixed to an introducer device 104. The malecot 100 is introduced with the outer sheath 102 in an undeployed, near-cylindrical configuration. As shown in FIG. 1B, the expandable malecot wing portion 106 of the outer sheath 102 is deployed by moving a proximal portion of the outer sheath 102 distally relative to the introducer device 104 and/or by moving the introducer device 104 proximally relative to a proximal portion of the outer sheath 102, which expands/folds outward the malecot wing portion 106. The introducer device 104 must be held in place in order to keep the malecot wing portion 106 deployed. Malecots commonly are constructed of a polymer or other material that may tend toward resuming a cylindrical configuration rather than retaining an expanded/deployed configuration. It would be advantageous to provide a malecot device that has an increased propensity for retaining its expanded/deployed configuration.

### BRIEF SUMMARY

In one aspect, embodiments of the present invention may include a malecot device that includes an elongate generally tubular body, which itself includes a proximal end and a distal end with an elongate body lumen disposed through its length. In one such embodiment, the body may include a plurality of generally parallel elongate slits defining lateral sides of a plurality of malecot wings, where the malecot wings are configured to deploy by folding outward from a central longitudinal axis of the body upon a deployment action moving the proximal and distal ends longitudinally toward each other, and where a first one of the malecot wings comprises at least a first magnet and a second magnet configured to engage each other when the first malecot wing is folded outward.

In another aspect, embodiments of the present invention may include a malecot device that includes at least one malecot wing, that itself includes a first magnet and a second magnet, where the first and second magnets are configured to engage each other and retain the malecot wing in a deployed configuration.

### BRIEF DESCRIPTION OF THE DRAWINGS

FIGS. 1A and 1B show, a prior art malecot device in, respectively, an undeployed and a deployed configuration;

FIG. 2A illustrates an external view of a malecot device embodiment;

FIGS. 2A and 2B depict, respectively, an undeployed and a deployed view of the malecot device of FIG. 2A, in longitudinal cross-section;

FIG. 2D shows an alternative embodiment of the malecot device of FIGS. 2A-2C in a deployed configuration, in longitudinal cross-section;

FIG. 3 depicts an external view of another malecot device embodiment;

FIGS. 4A-4E illustrate some examples of longitudinal cross-sectional geometries that may be used with a malecot device;

FIGS. 5A and 5B show, respectively, an external view and a cross-sectional view of another malecot device embodiment;

FIGS. 6A and 6B illustrate, respectively, an undeployed and a deployed view of a malecot device embodiment including an attached deployment device;

FIGS. 7A, 7B, and 7C depict, respectively, an undeployed and a deployed view of another malecot device embodiment including a removable deployment device, and a view showing removal of the deployment device;

FIGS. 8A, 8B, and 8C show, respectively, an undeployed and a deployed view of another malecot device embodiment including a removable deployment device, and a view showing removal of the deployment device; and

FIGS. 9A, and 9B depict, respectively, an undeployed view of another malecot device embodiment including a removable deployment device, and a view showing removal of the deployment device with the malecot deployed.

### DETAILED DESCRIPTION OF THE DRAWINGS AND VARIOUS EMBODIMENTS

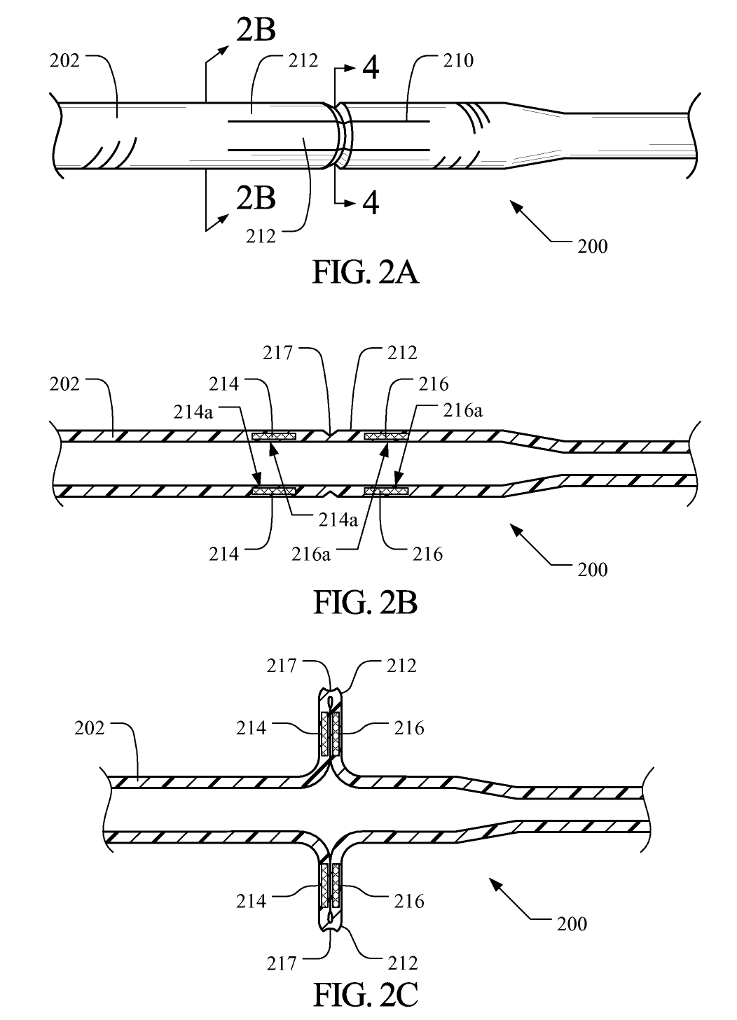

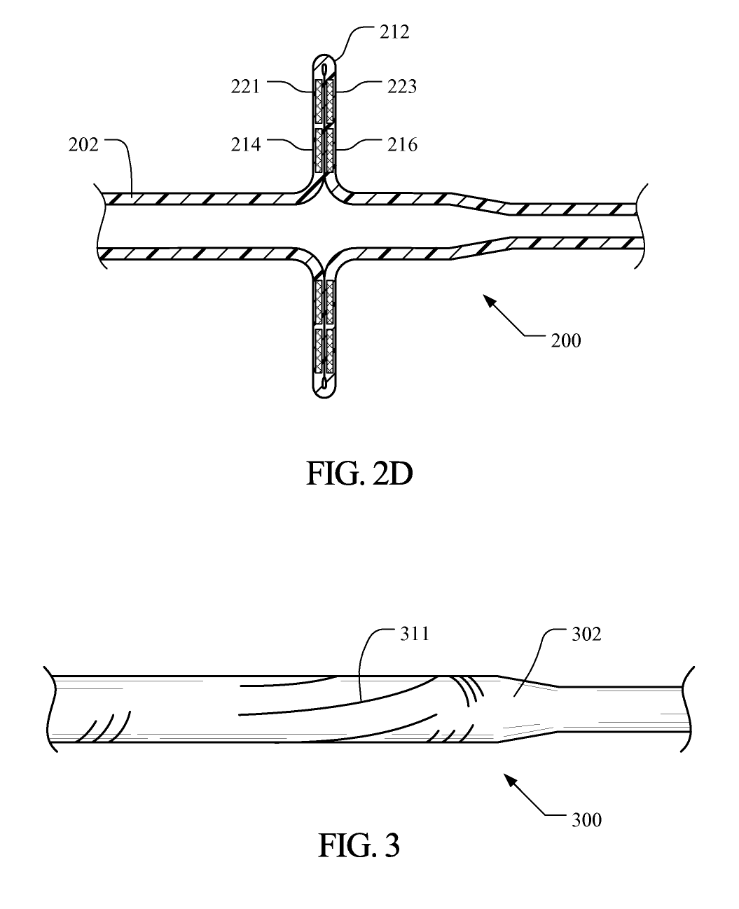

FIGS. 2A-2C show an embodiment of a malecot sheath 200 according to the present invention and having a generally tubular body configuration. FIG. 2A is an exterior view of a lengthwise portion of the malecot sheath 200 showing a generally parallel series of longitudinal slits 210 that define the lateral edges of a plurality malecot wings 212. The slits 210 are shown as being generally parallel to the longitudinal axis of the sheath 200, but those of skill in the art will appreciate that other sheath embodiments within the scope of the present invention may include a set of slits oriented at an angle or curve relative to the longitudinal axis, one example of which is shown in FIG. 3, where the slits are shown in a different orientation. Specifically, FIG. 3 shows an external view of a malecot sheath 300 with slits 311 through wall 302 disposed at an angle relative to a central longitudinal axis of the sheath 300.

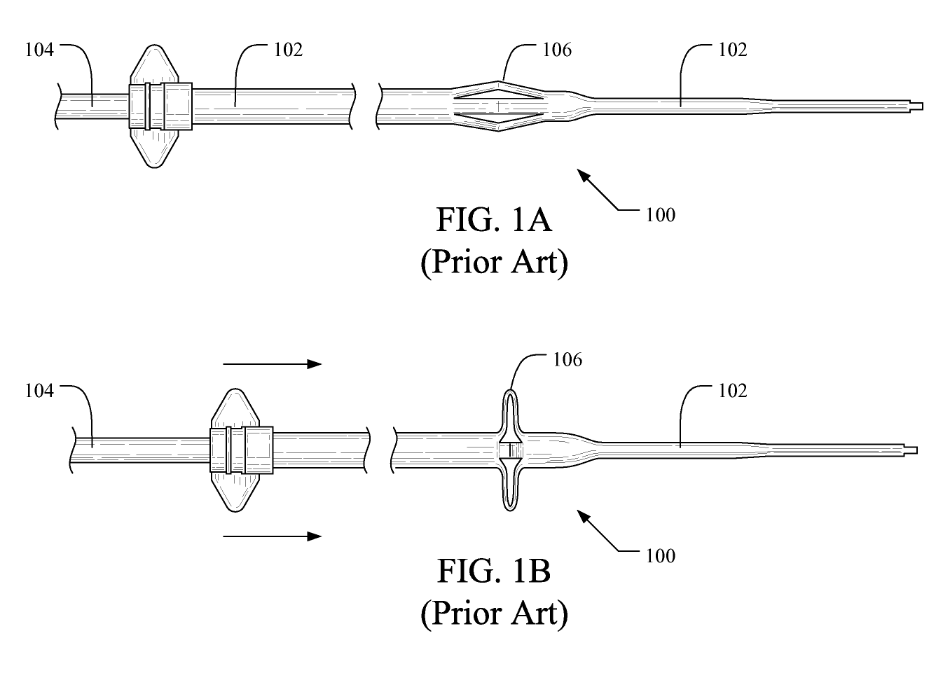

As shown in the longitudinal section view of FIG. 2B (taken along line 2B-2B of FIG. 2A), the malecot sheath 200 includes an outer wall 202 that preferably is constructed of a flexible polymer such as, for example, silicone, PTFE, polyurethane, or any other biocompatible material having physical characteristics suitable for use in a malecot device. The malecot wings 212 each include a first magnet 214 and a second magnet 216. The magnets are oriented such that a contact surface 214a of the first magnet 214 has the opposite polarity of, and will therefore be attracted toward, a contact surface 216a of the second magnet 216. FIG. 2C shows the malecot wings 212 deployed such that the first and second magnets 214, 216 are engaged and promote retention of the deployed configuration. Specifically, the magnets may hold malecot wings in a deployed configuration that resists any bias of the wings to unfold/undeploy—but without the need for holding a deployment member in place (or similar mechanisms) as was described with reference to FIGS. 1A and 1B.

As shown in FIG. 2C, the malecot wings 212 may include a scored or molded feature 217 on an inner or outer surface that may promote bending at a desired intermediate region rather than relying strictly upon folding in a larger generally intermediate region. Whether a scored or molded feature 217 is present or not, each malecot wing 212 may be considered as having a first wing portion 212a and a second wing portion 212b. The proximal and distal wing sections 212a, 212b may be separated by a predefined feature such as, for example, the scored or molded indentation 217 such that—during deployment of the malecot wings 212—they will fold along that feature in a hinged-type manner toward each other. In the absence of a scored or molded feature, the folding that occurs along a malecot wing to separate the proximal and distal wing sections 212a, 212b will generally be intermediate along a length of the malecot wing 212, but may not be at a predefined location. However, the proximal and distal magnets 214, 216 (located respectively in the proximal and distal wing sections 212a, 212b) preferably will attract each other with sufficient strength that an intermediate bend that may be considered as forming a border between the proximal and distal wing sections 212a, 212b will be located between the proximal and distal magnets.

FIG. 2D shows a different embodiment of the malecot sheath 200, which includes a third magnet 221 and a fourth magnet 223. The magnets are oriented such that a contact surface of the third magnet 221 has the opposite polarity of, and will therefore be attracted toward, a contact surface of the fourth magnet 223. The magnets may be incorporated into the body of the sheath 200 by use of an adhesive, overmolding, or any other suitable technique known to those of skill in the art, or developed in the future.

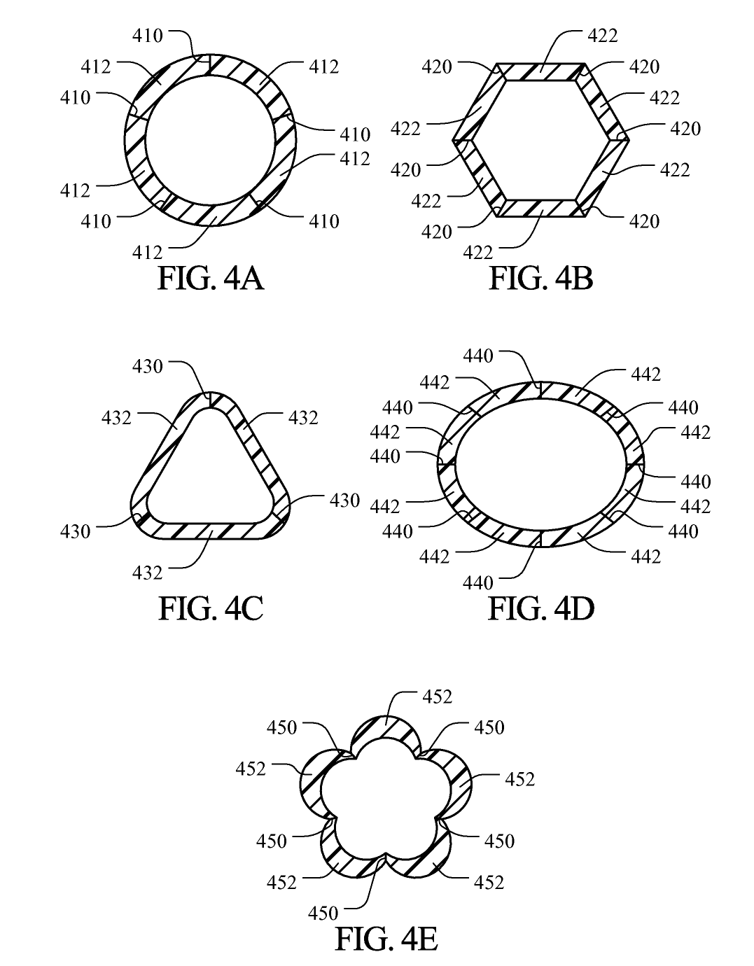

FIGS. 4A-4E show cross-sectional geometries of a malecot sheath that may be used within the scope of the present invention. Possible configurations include one or more of a cross-sectional geometry selected from circular, angled polygonal, rounded polygonal, and non-circular curvilinear. Specifically, each of FIGS. 4A-4E shows a cross-section from a perspective taken along line 4-4 of FIG. 2A that represents sample geometries (without providing an exhaustive list) that may be practiced within the scope of the present invention. Each of these is shown with the malecot wings in an undeployed configuration. FIG. 4A shows a circular cross-section having five slits 410 forming five malecot wings 412, although it should be appreciated that a malecot sheath of the present invention may include more or fewer slits and wings. FIG. 4B depicts a cornered/angled polygon embodied as hexagonal cross-section (which may be symmetrical or asymmetrical) having six slits 420 forming six malecot wings 422. FIG. 4C shows a rounded polygon embodied a “rounded triangle” cross-section having three slits 430 forming three malecot wings 432. FIG. 4D depicts a non-circular curvilinear cross-section as an ellipse having eight slits 430 forming eight malecot wings 432. FIG. 4E depicts another non-circular curvilinear cross-section as a “flower” having five slits 440 forming five malecot wings 442.

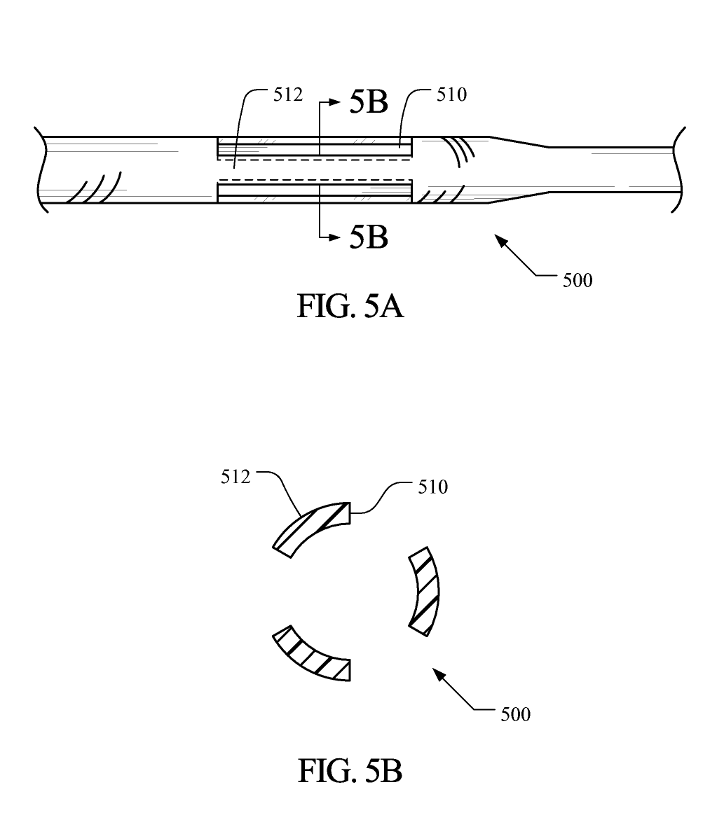

In some embodiments, and as shown in FIGS. 5A-5B, one or more of the slits that define the malecot wings may be wide enough to form apertures or slots therebetween. FIG. 5A shows an exterior side view of a malecot sheath 500 including wide apertures/slits 510 that define malecot wings 512, and FIG. 5B shows the same embodiment as a cross-sectional view taken along line 5B-5B of FIG. 5A.

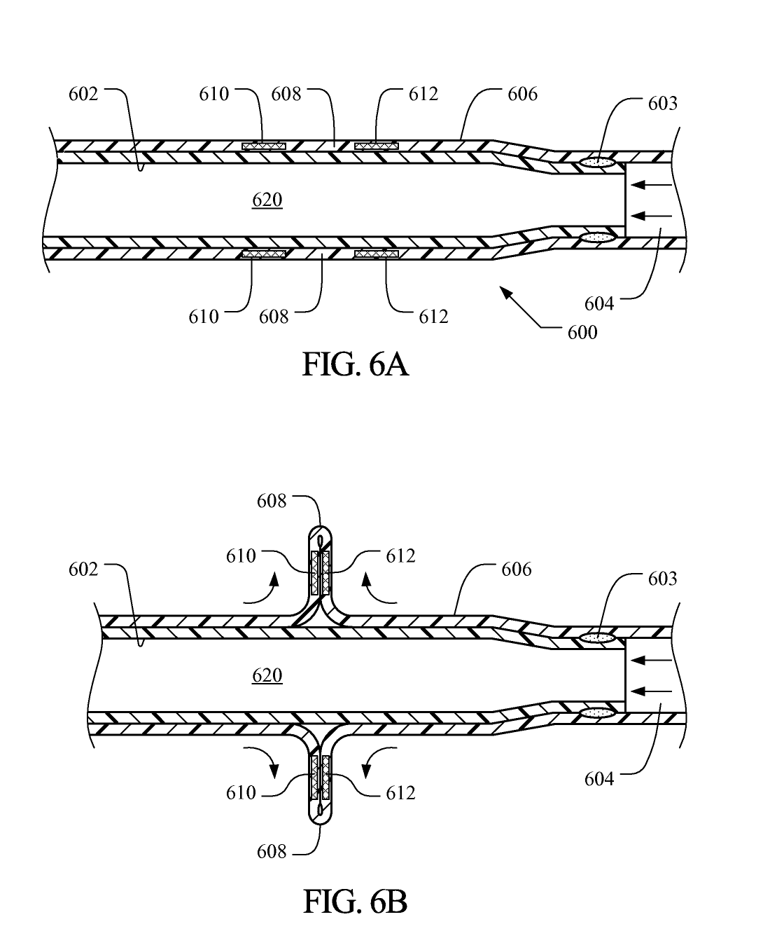

Various deployment mechanisms may be used with a malecot device of the present invention. FIGS. 6A and 6B show a malecot device 600 with a fixedly attached deployment member 602 disposed through a length of a sheath lumen 604 of a malecot sheath 606 and attached thereto at an attachment region 603. FIG. 6A shows the device 600 undeployed (e.g., in a configuration suitable for low-profile passage through a patient stoma prior to deployment). To deploy the malecot wings 608 in a manner allowing their proximal and distal magnets 610, 612 to engage, a user may move the deployment member 602 proximally relative to the sheath 606 and/or move a proximal region of the sheath 606 distally relative to the deployment member 602. As shown in FIG. 6B, the resulting foreshortening/longitudinal compression of the sheath 606 deploys the malecot wings 608. When it becomes desirable to remove the device 600, the process is simply reversed to collapse the malecot wings 608 into the original lower profile aspect. This configuration provides a patent lumen 620 through the deployment member 602.

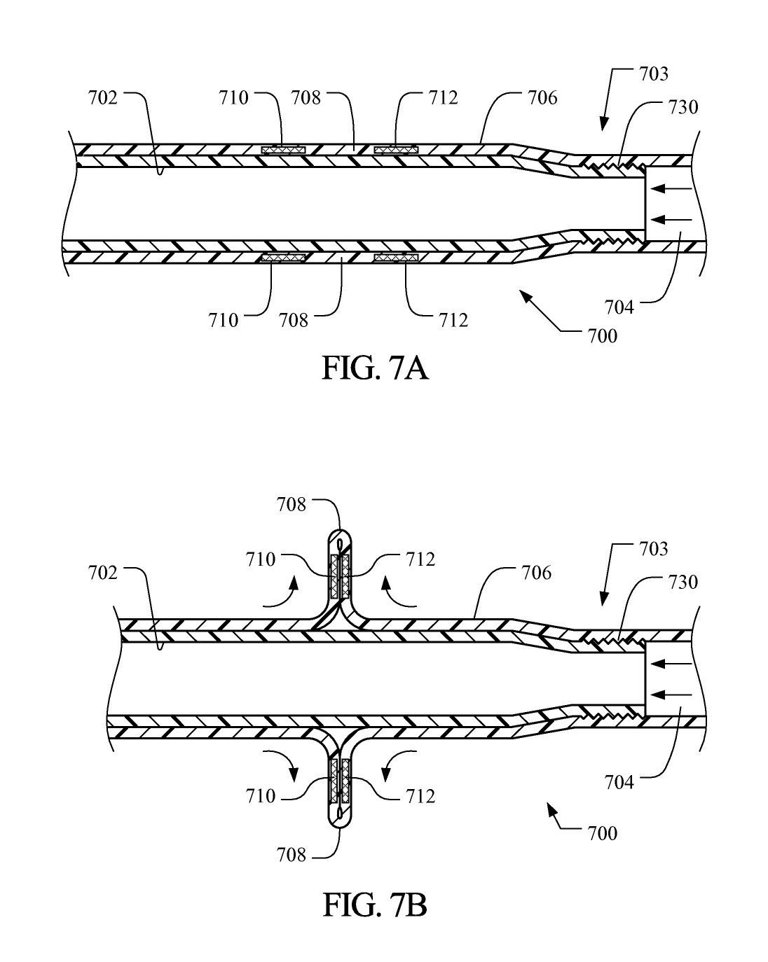

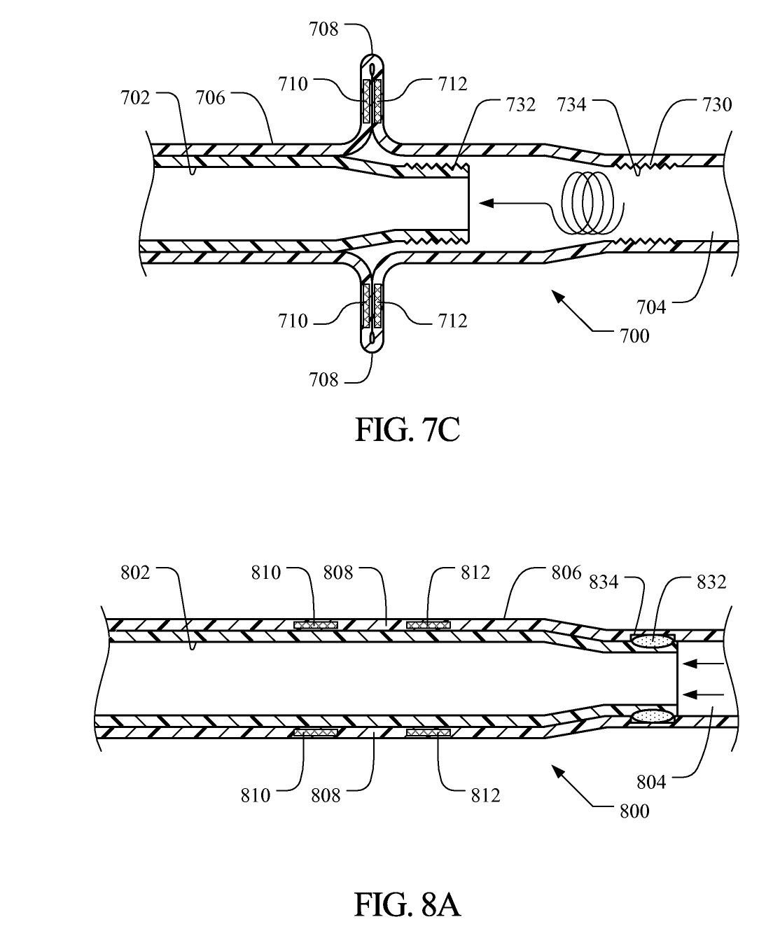

One of the challenges of existing malecot devices is to provide a malecot device with a small outer diameter in order to provide a desirable interface with a patient (e.g., requiring a smaller stoma for an enteral feeding tube or drainage catheter), while maintaining a desirably large inner diameter to facilitate efficient passage of material through the generally tubular body. This may be effected in one manner by providing a removable deployment member. FIGS. 7A through 7C show a malecot device 700 with a removable deployment member 702. As shown in FIG. 7A, the deployment member 702 is disposed through a length of a sheath lumen 704 of a malecot sheath 706 and attached removably thereto at an attachment location 703 by an attachment interface 730. The attachment interface 730 is shown as a threaded interface with a first threaded surface 732 on the outer surface of the deployment member 702 engaging a complementary second threaded surface 734 on the inner surface of the sheath lumen 704. Those of skill in the art will appreciate that, for example, a bayonet mechanism, hook and loop connection, or other attachment means may be used in lieu of a threaded interface, while remaining within the scope of the present invention.

FIG. 7A shows the device 700 undeployed (e.g., in a configuration suitable for low-profile passage through a patient stoma prior to deployment). To deploy the malecot wings 708 in a manner allowing their proximal and distal magnets 710, 712 to engage, a user may move the deployment member 702 proximally relative to the sheath 706 and/or move a proximal region of the sheath 706 distally relative to the deployment member 702. As shown in FIG. 7B, the resulting foreshortening/longitudinal compression of the sheath 706 deploys the malecot wings 708. The deployment member 702 may be removed, preferably by holding the sheath 706 in place and rotatingly disengaging the threaded surfaces 732, 734. When it becomes desirable to remove the device 700, the process is simply reversed to collapse the malecot wings 708 into the original lower profile aspect.

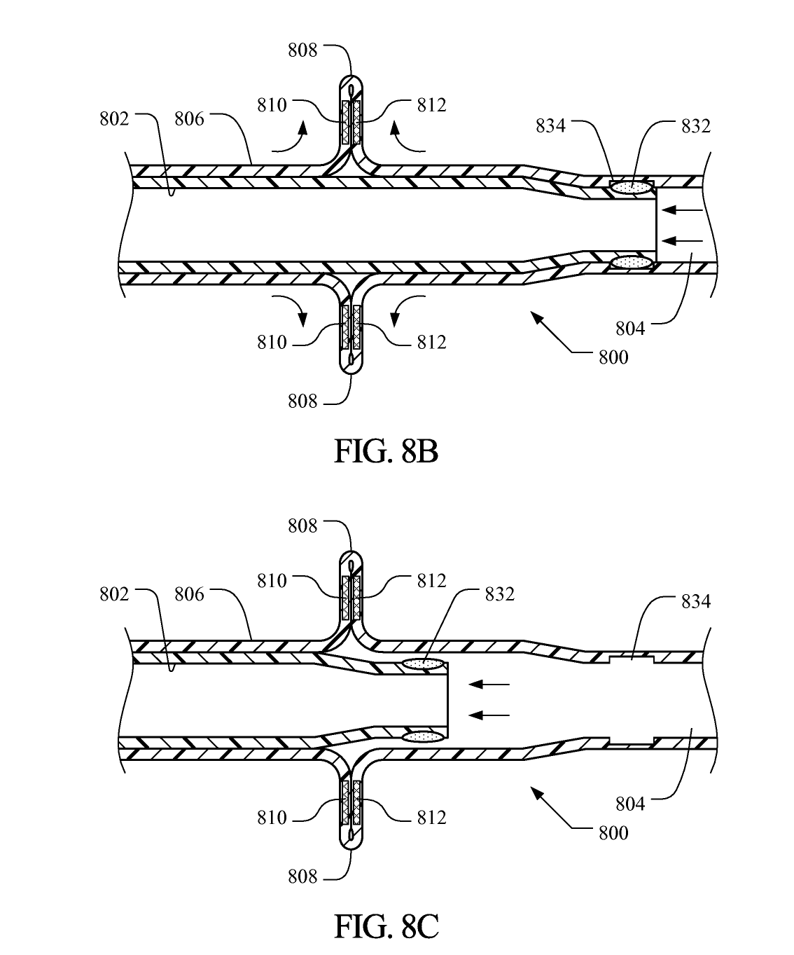

FIGS. 8A through 8C show another embodiment of a malecot device 800 with a removable deployment member 802. As shown in FIG. 8A, the deployment member 802 is disposed through a length of a sheath lumen 804 of a malecot sheath 806 and attached removably thereto at an attachment location by an attachment interface 830. The attachment interface 830 is shown as an expandable member 832, embodied here as a circumferential balloon, in an expanded configuration, on the outer surface of the deployment member 802 frictionally engaging a surface 834 on the inner surface of the sheath lumen 804, which is shown as including a complementarily indented surface. Those of skill in the art will appreciate that embodiments not including an embedded surface and/or including other features such as texturized surfaces to enhance friction may be used within the scope of the present invention. Likewise, the expandable member may be embodied as a balloon, a wire basket, or another expandable member currently known or yet to be developed in the art, while being practiced within the scope of the present invention. Additionally, because the deployment member is removable, a portion of its inner diameter can easily be occupied by actuation means of an expandable member such as, for example, an inflation lumen for a balloon, a drive wire for a basket or the like, or a drive shaft configured for actuation of an expandable anchor or jaw mechanism.

FIG. 8A shows the device 800 undeployed (e.g., in a configuration suitable for low-profile passage through a patient stoma prior to deployment). To deploy the malecot wings 808 in a manner allowing their proximal and distal magnets 810, 812 to engage, a user may move the deployment member 802 proximally relative to the sheath 806 and/or move a proximal region of the sheath 806 distally relative to the deployment member 802. As shown in FIG. 8B, the resulting foreshortening/longitudinal compression of the sheath 806 deploys the malecot wings 808. The deployment member 802 may be removed, preferably by reducing the outer diameter of the expandable member 832 (e.g., at least partially deflating, if a balloon; at least partially retracting if a basket; etc.). When it becomes desirable to remove the device 800, the process is simply reversed to collapse the malecot wings 808 into the original lower profile aspect.

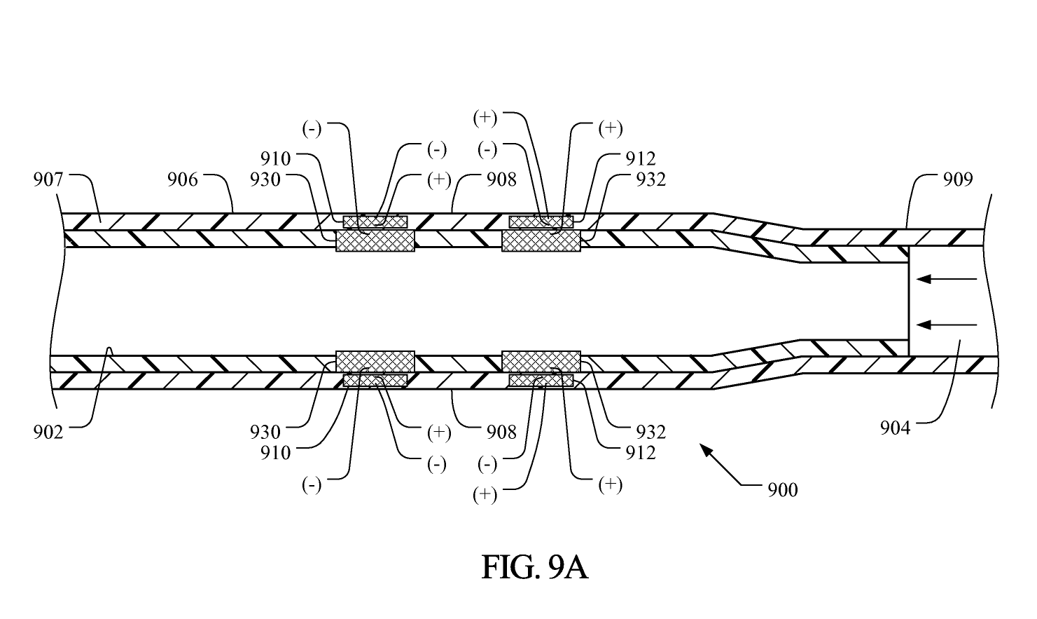

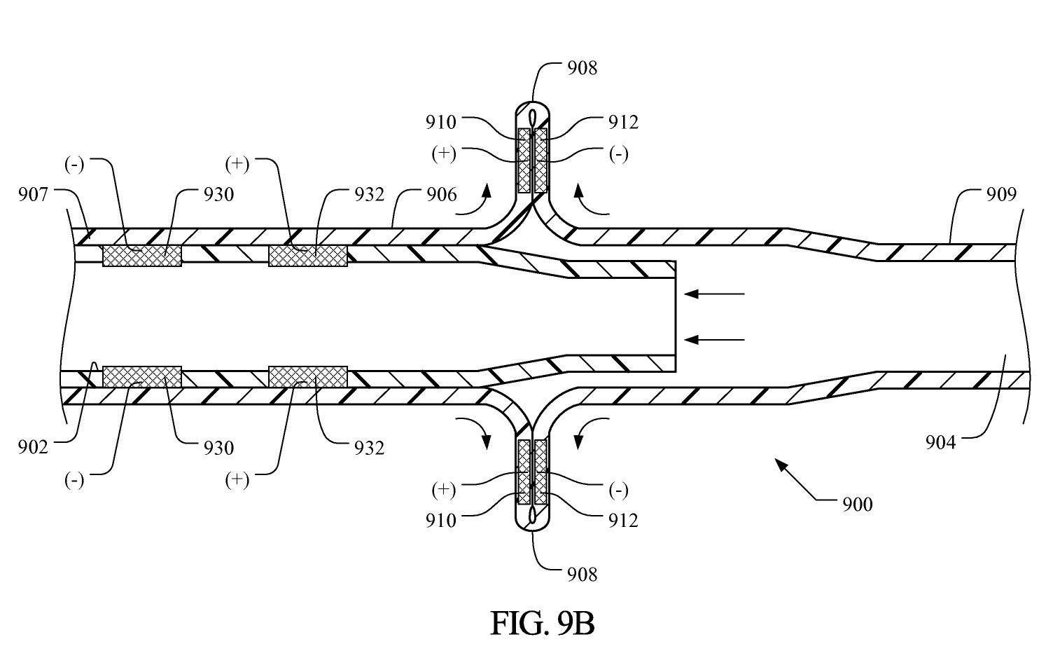

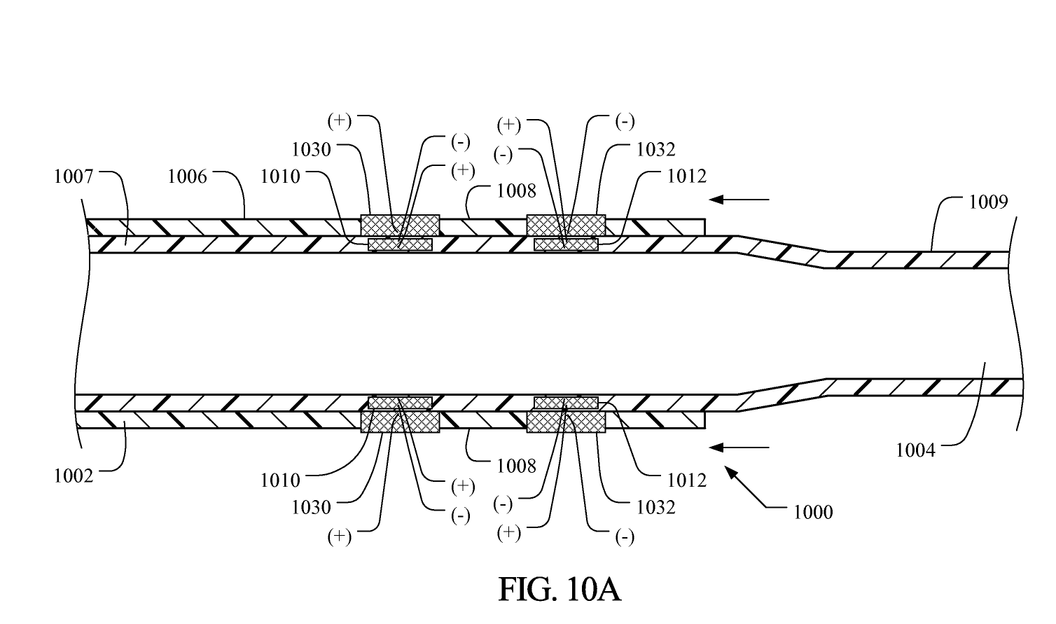

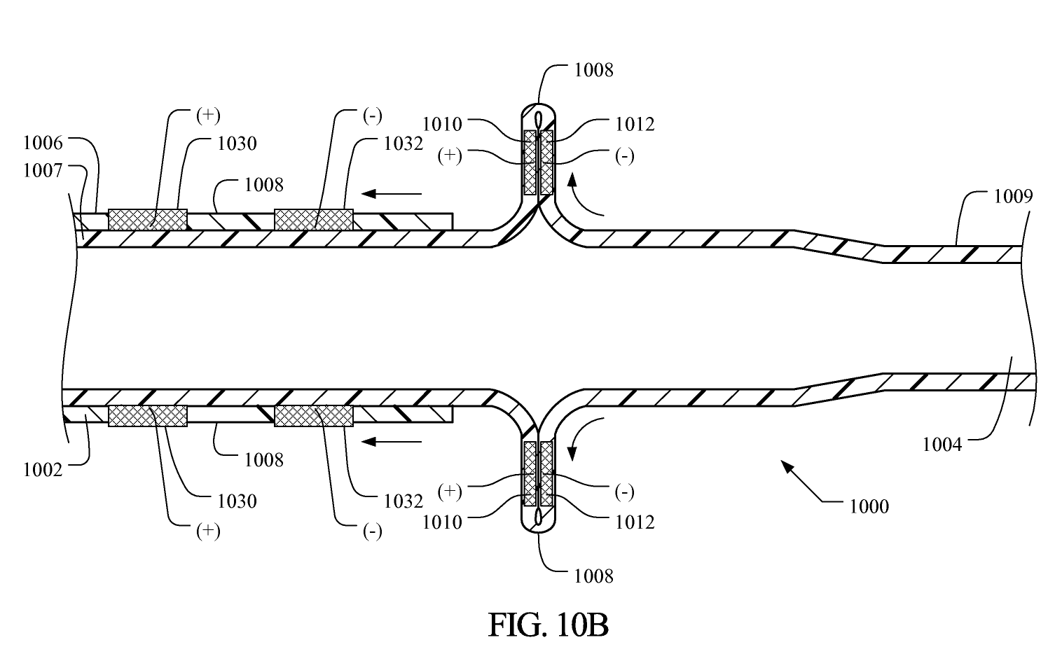

FIGS. 9A through 9B show a further embodiment of a malecot device 900 with an internal removable deployment member 902. The malecot device 900 includes a malecot sheath 906 with a proximal catheter portion 907 and a distal catheter portion 909 configured to provide a path of fluid communication. A deployment member 902 is disposed through a length of a sheath lumen 904 of the malecot sheath 906 and attached removably thereto at an attachment location by an attachment interface 930. The attachment interface 930 includes a distal plurality of deployment magnets 932 configured for engaging distal malecot wing magnets 912, and a proximal plurality of deployment magnets 930 configured for engaging proximal malecot wing magnets 910. The polarity of each magnet's contact surface is indicated with a plus sign (+) or minus sign (−), where opposite polarities attract each other and like polarities repel each other.

FIG. 9A shows the device 900 in a low-profile, undeployed configuration (e.g., in a configuration suitable for low-profile passage through a patient stoma prior to deployment) where the proximal deployment magnets 930 are engaged with the proximal malecot wing magnets 910, and the distal deployment magnets 932 are engaged with the distal malecot wing magnets 912. To deploy the malecot wings 908 in a manner allowing their proximal and distal malecot wing magnets 910, 912 to engage each other, a user may move the deployment member 902 proximally relative to the sheath 906 and/or move the proximal region of sheath 906 distally relative to the deployment member 902.

As shown in FIG. 9B, proximal movement of the deployment member 902 relative to the sheath 906 effects deployment of the malecot wings 908. Specifically, this movement causes the distal deployment magnets 932 to attract/pull the distal malecot wing magnets 912 generally proximally, while simultaneously repelling the proximal malecot wing magnets 910, thereby deploying the malecot wings 908 and allowing/promoting the engagement of the proximal and distal malecot wing magnets 910, 912 with each other. This action may be enhanced by use of distal deployment magnets 932 having greater magnetic force than the malecot wing magnets 910, 912. It should be appreciated that a deployment member 902 may be implemented within the scope of the present invention with only one of a proximal and/or distal deployment magnet. The deployment member 902 may subsequently be completely removed. When it becomes desirable to reduce the outer diameter of and remove the device 900, the process may simply be reversed to collapse the malecot wings 908 into the original lower profile aspect.

FIGS. 10A through 10B show still another embodiment of a malecot device 1000 with an external removable deployment member 1002. The malecot device 1000 includes a malecot sheath 1006 with a proximal catheter portion 1007 and a distal catheter portion 1009 configured to provide a path of fluid communication through a sheath lumen 1004. A deployment member 1002 is disposed around the outside of the malecot sheath 1006 and attached removably thereto at an attachment location by an attachment interface 1030. The attachment interface 1030 includes a distal plurality of deployment magnets 1032 configured for engaging distal malecot wing magnets 1012, and a proximal plurality of deployment magnets 1030 configured for engaging proximal malecot wing magnets 1010. The polarity of each magnet's contact surface is indicated with a plus sign (+) or minus sign (−), where opposite polarities attract each other and like polarities repel each other.

FIG. 10A shows the device 1000 in a low-profile, undeployed configuration (e.g., in a configuration suitable for low-profile passage through a patient stoma prior to deployment) where the proximal deployment magnets 1030 are engaged with the proximal malecot wing magnets 1010, and the distal deployment magnets 1032 are engaged with the distal malecot wing magnets 1012. To deploy the malecot wings 1008 in a manner allowing their proximal and distal malecot wing magnets 1010, 1012 to engage each other, a user may move the deployment member 1002 proximally relative to the sheath 1006 and/or move the proximal region of sheath 1006 distally relative to the deployment member 1002. In a preferred aspect of this embodiment, the malecot wings 1018 will be biased outward into a deployed configuration.

As shown in FIG. 10B, proximal movement of the deployment member 1002 relative to the sheath 1006 allows deployment of the malecot wings 1008. Specifically, as the deployment member 1002 is withdrawn from around the malecot wings 1008, the attraction of the proximal and distal malecot magnets 1030, 1032 toward each other (potentially enhanced by an outward/deployed bias of the malecot wings) will deploy the malecot wings 1018. it should be appreciated that a deployment member 1002 may be implemented within the scope of the present invention with only one of a proximal and/or distal deployment magnet, or with no magnets at all (in which embodiment, a frictional surface contact between the inner surface of the deployment member 1002 and the outer surface of the malecot sheath 1006 may be used to keep them together until deployment). The deployment member 1002 may subsequently be completely removed. When it becomes desirable to reduce the outer diameter of and remove the device 1000, the process may simply be reversed to collapse the malecot wings 1008 into the original lower profile aspect (or, an inner deployment member such as, for example, of the type described with reference to FIGS. 9A-9B or another, may be used to effect undeployment and removal).

It should be appreciated that the attachment and removability features of the deployment devices described here may be used alone or in combination, all within the scope of the present invention. Magnetic means and devices may also be used in other malecot devices, without departing from the scope of the present invention. Many other modifications and other aspects of the invention may come to mind to one skilled in the art to which this invention pertains having the benefit of the teachings presented in the foregoing description; and it will be apparent to those skilled in the art that variations and modifications of the present invention can be made without departing from the scope or spirit of the invention. Accordingly, it should be understood that the invention is not to be limited to the specific aspects disclosed and that modifications and other aspects are intended to be included within the scope of the appended claims. Although specific terms are employed herein, they are used in a generic and descriptive sense only and not for purposes of limitation.

1. A malecot device comprising:
an elongate generally tubular body including a proximal end and a distal end with an elongate body lumen disposed through its length;
where the body includes a plurality of generally parallel elongate slits defining lateral sides of a plurality of malecot wings, and each malecot wing has a proximal wing portion and a distal wing portion;
where the malecot wings are configured to deploy by folding outward from a central longitudinal axis of the body upon a deployment action moving the proximal and distal body ends longitudinally toward each other; and
where at least one of the malecot wings comprises at least a first magnet in one of its proximal or distal wing portion and a second magnet in the other of its proximal or distal wing portion, and the first and second magnets are configured to engage each other when the first malecot wing is folded outward.

2. The malecot device of claim 1, where the slits are oriented generally parallel with a central longitudinal axis of the body.

3. The malecot device of claim 1, where the slits are oriented generally at an angle relative to a central longitudinal axis of the body.

4. The malecot device of claim 1, where at least one of the malecot wings comprises:
a third magnet and a fourth magnet that are configured to engage each other when the at least one malecot wing is folded outward.

5. The malecot device of claim 1, where the elongate tubular body includes a cross-sectional geometry selected from circular, angled polygonal, rounded polygonal, and non-circular curvilinear.

6. The malecot device of claim 1, further comprising:
at least one of a score and a molded feature on a surface of the body configured to promote bending in the immediate region of the feature.

7. The malecot device of claim 6, where the at least one of a score and a molded feature is disposed in a location selected from the group consisting of:
adjacent a proximal end of a malecot wing,
adjacent a distal end of a malecot wing,
between proximal and distal ends of a malecot wing,
on a lumen-facing surface;
on an outside-facing surface; and
any combination thereof.

8. The malecot device of claim 1, where each of the plurality of malecot wings includes a magnet.

9. The malecot device of claim 1, further comprising an elongate deployment member configured for deployment of the device.

10. The malecot device of claim 1, further comprising:
an elongate deployment member disposed coaxially through at least a portion of the elongate body lumen and accessible from the proximal end of the body.

11. The malecot device of claim 10, further comprising:
the deployment member being fixedly attached to the body in at least one attachment location that is distal of the malecot wings; and
a length of the deployment member proximal of the attachment location being slidably disposed relative to the body, such that a proximal movement of the deployment member relative to the body will deploy the malecot wings.

12. The malecot device of claim 11, further comprising:
the deployment member being removably attached to the body in at least one attachment location that is distal of the malecot wings; and
a length of the deployment member proximal of the attachment location being slidably disposed relative to the body, such that a proximal movement of the deployment member relative to the body will deploy the malecot wings.

13. The malecot device of claim 12, where the deployment member further comprises:
at least a third magnet oriented to engage one of the first magnet and the second magnet.

14. The malecot device of claim 13, where the deployment member further comprises:
at least a fourth magnet oriented to engage the other of the first magnet and the second magnet.

15. The malecot of claim 12, where the removable attachment comprises a threaded connection.

16. The malecot of claim 12, where the deployment member comprises an expandable member configured to releasably engage an inner surface of the body.

17. A malecot device comprising:
at least one malecot wing, the at least one malecot wing including a first magnet and a second magnet, where the first and second magnets are configured to engage each other and retain a deployed configuration of the malecot wing.

18. The malecot device of claim 17, further comprising:
at least one catheter portion.

19. The malecot device of claim 17, further comprising:
a deployment member.

20. The malecot device of claim 18, where the deployment member is removable.
